(19) United States
(12) Patent Application Publication
(10) Pub. No.: US 2008/0070949 A1
(43) Pub. Date: Mar. 20, 2008

(54) POLYMORPHS OF RIMONABANT

(75) Inventors: Manjinder Singh, Mumbai (IN); Dharmaraj Ramachandra Rao, Mumbai (IN); Rajendra Narayanrao Kankan, Mumbai (IN)

Correspondence Address: CONLEY ROSE, P.C., 5601 GRANITE PARKWAY, SUITE 750, PLANO, TX, 75024, US

(73) Assignee: Cipla Limited, Maharashtra (IN)

(21) Appl. No.: 11/736,110

(22) Filed: Apr. 17, 2007

(30) Foreign Application Priority Data
Sep. 19, 2006 (IN) 1491/MUM/2006
Sep. 19, 2006 (IN) 1492/MUM/2006

(51) Int. Cl. A61K 31/4523; A61P 25/34; A61P 3/04; C07D 401/12

(52) U.S. Cl. 514/326; 546/211

(57) ABSTRACT

Crystalline form C of rimonabant and amorphous rimonabant, processes for their preparation and pharmaceutical compositions thereof.

### CROSS-REFERENCE TO RELATED APPLICATIONS

This application claims priority of Indian Patent Application Nos. 1491/MUM/2006 entitled “Amorphous Form of Rimonabant, Process for Preparation and Compositions Thereof” and 1492/MUM/2006 entitled “Crystalline Form of Rimonabant, Process for Preparation and Compositions Thereof,” both filed Sep. 19, 2006, which applications are incorporated by reference herein in their entirety.

### FIELD OF THE INVENTION

The present invention relates to a new crystalline form of N-piperidino-5-(4-chlorophenyl)-1-(2,4-dichlorophenyl)-4-methyl-3-pyrazolecarboxamide, a process for its preparation and pharmaceutical compositions thereof. The present invention also relates to an amorphous form of N-piperidino-5-(4-chlorophenyl)-1-(2,4-dichlorophenyl)-4-methyl-3-pyrazolecarboxamide, a process for its preparation and pharmaceutical compositions thereof.

### BACKGROUND OF THE INVENTION

Rimonabant (Formula I) is an antagonist of the CB 1 cannabinoid receptors and is chemically termed as N-piperidino-5-(4-chlorophenyl)-1-(2,4-dichlorophenyl)-4-methyl-3-pyrazole carboxamide.

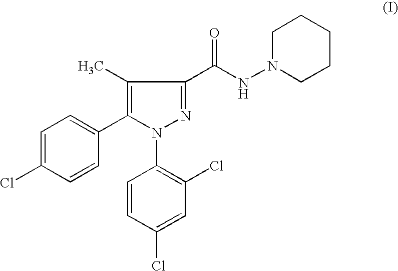

European patent EP0656354 disclosed rimonabant and its pharmaceutical acceptable salts for the first time. EP0656354 makes no reference to the existence of any specific polymorphic forms of rimonabant. The process disclosed in this patent results in rimonabant in crystalline form, which form is termed as crystalline Form I in subsequent patents.

US patent application US20050043356 discloses a new crystalline form of rimonabant named as Form II.

WO2006087732 discloses an amorphous form, and crystalline forms of Form II, Form III and Form IV of rimonabant hydrochloride, processes for their preparation and pharmaceutical compositions containing the same.

It is reported and we have confirmed that crystalline rimonabant has a tendency to form a solvate with methanol, ethanol, isopropyl alcohol and various other organic solvents such as ethyl acetate, tetrahydrofuran, methyl cyclohexane, hexane, heptane, isopropyl acetate, acetone, acetonitrile, methyl ethyl ketone, dimethyl formamide, dimethyl acetamide. In crystalline rimonabant the solvent gets trapped in the crystal's lattice and hence removal of these residual solvent becomes difficult. The solvated form of any drug is not acceptable to any regulated authority for any pharmaceutical use. Further, to get rid of the residual solvent, purification or crystallization is required, which leads to a loss in yield. Alternatively, the solvate has to be dried at elevated temperatures for a prolonged time. Other desolvating techniques known in the art include suspending the solvate in an anti-solvent and digesting for extended periods.

It is well known that different polymorphic forms of the same drug may have substantial differences in certain pharmaceutically important properties. Furthermore, different physical forms may have different particle size, hardness and glass transition temperatures.

However, it is well known in the art that the existence of polymorphic forms of any given compound cannot be predicted, and there is no standard procedure for proceeding to make a previously unknown polymorphic form. Even after a polymorph has been identified, there is no possibility of predicting whether any additional forms will ever be discovered. This has been described in many recent articles, including A. Goho, Science News, Vol. 166, No. 8, pages 122-123 (August 2004).

Consequently, it would be a significant contribution to the art to provide a new crystalline form of rimonabant, methods of preparation, pharmaceutical formulations, and methods of use thereof.

Amorphous and crystalline forms of a drug may have different handling properties, dissolution rates, solubility, and stability.

Amorphous materials do not exhibit the three-dimensional long-range orders found in crystalline materials, but are structurally more similar to liquids where the arrangement of molecules is random.

Amorphous solids do not give a definitive x-ray diffraction pattern (XRD). In addition, amorphous solids do not give rise to a specific melting point and tend to liquefy at some point beyond the glass transition temperature. Because amorphous solids do not have lattice energy, they usually dissolve in a solvent more rapidly and consequently may provide enhanced bioavailability characteristics such as a higher rate and extent of absorption of the compound from the gastrointestinal tract. Also, amorphous forms of a drug may offer significant advantages over crystalline forms of the same drug in the manufacturing process of solid dosage form such as compressibility.

Consequently, it would be a significant contribution to the art to provide an amorphous form of rimonabant having increased solubility, processes for its preparation, pharmaceutical formulations thereof, and methods of use thereof.

The present invention provides a new crystalline form of rimonabant and a process for preparation of the same. The advantages of the process include simplicity, eco-friendliness and suitability for commercial use. Another major advantage is that the present invention provides rimonabant substantially free of solvent contamination.

The present invention also provides amorphous rimonabant and a process for preparing it. The advantages of the process include simplicity, eco-friendliness and suitability for commercial use. The known processes for making amorphous substances include lyophilization and spray drying. These are, however, time consuming and expensive. The process of the present invention is surprisingly very simple, economical and eco-friendly.

### SUMMARY OF THE INVENTION

According to a first aspect of the present invention, there is provided a new crystalline form of rimonabant hereinafter termed as Form C. Crystalline Form C of rimonabant is characterised by having an XRPD pattern with characteristic °2θ peaks at about 8.8, 14.5, 14.8, 21.6, 23.2, 23.6, 24.5 and 29.9±0.2 °2θ. In an embodiment, the Form C rimonabant has an XRPD pattern with further °2θ peaks at 15.1 and 19.1±0.2 °2θ. In a further embodiment, the Form C rimonabant has an XRPD pattern with further °2θ peaks at 9.3, 10.4, 13.4, 16.1, 16.2, 17.0, 17.7, 18.9, 19.5, 20.3, 20.7, 21.1, 22.4, 22.8, 23.7, 25.2, 27.2, 27.7, 29.5 and 30.5±0.2 °2θ.

According to another aspect of the present invention, there is provided crystalline Form C of rimonabant characterised by having an IR spectrum with characteristic peaks at 3639 cm⁻¹, 3388 cm⁻¹, 3207 cm⁻¹, 3079 cm⁻¹, 2806 cm⁻¹, 1556 cm⁻¹, 1265 cm⁻¹, 1138 cm⁻¹, 918 cm⁻¹, 634 cm⁻¹±2 cm⁻¹.

According to another aspect of the present invention, there is provided crystalline Form C of rimonabant characterised by having a moisture content ranging from 1% to 5%.

In an embodiment, crystalline Form C of rimonabant has an XRPD pattern, or substantially the same XRPD pattern, as set out in FIG. 1.

In another embodiment, crystalline Form C of rimonabant has an IR spectrum, or substantially the same IR spectrum, as set out in FIG. 2.

In a still further embodiment, crystalline Form C of rimonabant has a moisture content ranging from 3.0% to 4.5%. Suitably, the Form C rimonabant has a moisture content ranging from 3.5% to 4.5%.

According to another aspect of the present invention, there is provided a process for the preparation of crystalline rimonabant Form C. The process comprises the steps of: (a) using an acid to convert rimonabant to an acid addition salt of rimonabant; (b) dissolving the acid addition salt of rimonabant in a water miscible solvent; (c) adding a base to the solution; (d) and isolating crystalline Form C of rimonabant.

According to another aspect of the present invention, there is provided a process for preparing crystalline Form C of rimonabant comprising the steps of: (a) dissolving rimonabant in a water miscible solvent; (c) adding a base to the solution; (d) and isolating crystalline Form C of rimonabant.

In an embodiment, the base is an inorganic base. Suitably, the inorganic base is selected from the group consisting of sodium hydroxide, ammonium hydroxide, potassium hydroxide, sodium carbonate, sodium bicarbonate, potassium bicarbonate and potassium carbonate.

In an embodiment, the water miscible solvent is selected from the group consisting of a C1 to C6 straight- or branched-chain alcohol, acetone and acetonitrile. Suitably, the water miscible solvent is methanol or ethanol.

According to another aspect of the present invention, there is provided a process for preparing crystalline Form C of rimonabant comprising the steps of: (a) converting rimonabant to an acid addition salt of rimonabant using the corresponding acid; (b) suspending the acid addition salt of rimonabant in a solution of water and a surfactant; (c) adding a base to the suspension; (d) and isolating crystalline Form C of rimonabant.

Suitably, the base is selected from the group consisting of sodium hydroxide, potassium hydroxide, sodium carbonate, sodium bicarbonate, potassium bicarbonate and potassium carbonate.

Typically, the surfactant is selected from the group consisting of macrogol esters, polysorbates 20, 40, 60, 80 and 85, mono- and diglycerides of C12-C18 fatty acids, C₂-C₂₀ polyhydric alcohols, glycerin, propylene glycol, polyethylene glycol, ethylene glycol dimethyl ether, tetraethylene glycol dimethyl ether, triacetin, medium chain (C₆-C₁₀) triglycerides, and polyoxyethylene sorbitan monoesters. In an embodiment, the surfactant is present in a catalytic amount.

In another embodiment, the acid is selected from hydrochloric acid, oxalic acid, mandelic acid, tartaric acid, citric acid, salicylic acid, fumaric acid, sulphuric acid and phosphoric acid.

The crystalline Form C of rimonabant may be converted to a pharmaceutically acceptable salt of rimonabant, preferably the hydrochloride salt. Any conventional process may be used for the conversion, for example one of the processes disclosed in EP0656354.

Crystalline Form C of rimonabant prepared according to any one of the above processes forms another aspect of the present invention.

The crystalline rimonabant Form C of the present invention is stable and has good flow characteristics.

According to another aspect of the present invention, there is provided amorphous rimonabant. “Amorphous rimonabant” may also be termed “rimonabant free base in amorphous form” or “rimonabant in amorphous form.”

According to a further aspect of the present invention, there is provided amorphous rimonabant, characterised by having an IR spectrum with characteristic peaks at 3407 cm⁻¹, 3305 cm⁻¹, 3144 cm⁻¹, 3049 cm⁻¹, 2937 cm⁻¹, 2803 cm⁻¹, 1442 cm⁻¹, 1409 cm⁻¹, 1245 cm⁻¹ and 863 cm⁻¹, 2 cm⁻¹.

In an embodiment, the amorphous rimonabant has an XRPD pattern, or substantially the same XRPD pattern, as set out in FIG. 3.

In another embodiment, the amorphous rimonabant has an IR spectrum, or substantially the same IR spectrum, as set out in FIG. 4.

According to another aspect of the present invention, there is provided a process for the preparation of amorphous rimonabant comprises the steps of: (a) using an acid to convert rimonabant to an acid addition salt of rimonabant; (b) suspending the acid addition salt of rimonabant in water; (c) adding a base to the suspension; (d) and isolating amorphous rimonabant.

In an embodiment, steps (b) and (c) are carried out in the absence of a surfactant.

In an embodiment, the base is aqueous ammonia.

The amorphous form of rimonabant may be converted to a pharmaceutically acceptable salt of rimonabant, preferably the hydrochloride salt. Any conventional process may be used for the conversion, for example one of the processes disclosed in EP0656354.

Amorphous rimonabant prepared according to the process of the present invention forms another aspect of the present invention.

According to another aspect of the present invention, there is provided a pharmaceutically acceptable salt of rimonabant prepared according the processes described above.

According to another aspect of the present invention, there is provided a pharmaceutical composition comprising: crystalline Form C of rimonabant, amorphous rimonabant, or a pharmaceutically acceptable salt of rimonabant prepared according to the processes described above; and one or more pharmaceutical excipients.

According to another aspect of the present invention, there is provided the use of crystalline Form C of rimonabant, amorphous rimonabant, or a pharmaceutically acceptable salt of rimonabant prepared according to the processes described above in medicine.

According to another aspect of the present invention, there is provided the use of crystalline Form C of rimonabant, amorphous rimonabant, or a pharmaceutically acceptable salt of rimonabant prepared according to the processes described above in the manufacture of a medicament for treating weight management disorders and smoking addiction. For example, the weight management disorder may be obesity.

According to another aspect of the present invention, there is provided a method of treating weight management disorders or smoking addiction in a patient in need of such treatment, which method comprises administering to the patient a therapeutically effective amount of crystalline Form C of rimonabant, amorphous rimonabant, or a pharmaceutically acceptable salt of rimonabant prepared according to the processes described above. For example, the weight management disorder may be obesity.

According to another aspect of the present invention, there is provided a pharmaceutical composition that comprises crystalline rimonabant form C and one or more pharmaceutically acceptable carriers.

### BRIEF DESCRIPTION OF THE DRAWINGS

FIG. 1 is an X-ray powder diffraction (XRPD) pattern for crystalline rimonabant Form C.

FIG. 2 is an Infra red (IR) spectrum for crystalline rimonabant Form C.

FIG. 3 is an X-ray powder diffraction (XRPD) pattern for amorphous rimonabant.

FIG. 4 is an Infra red (IR) spectrum for amorphous rimonabant.

FIG. 5 is a graph of intrinsic dissolution of Form I, Form C and amorphous rimonabant by an HPLC-UV method.

### DETAILED DESCRIPTION

All technical and scientific terms used herein have the same meaning as commonly understood by one of ordinary skill in the art unless defined otherwise. For purposes of the present invention, the following terms are defined below.

The term “pharmaceutical composition” is intended to encompass a product comprising the active ingredient(s) and pharmaceutically acceptable excipients, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing the active ingredient, any additional active ingredient(s), and pharmaceutically acceptable excipients.

The term “pharmaceutically acceptable” means that which is useful in preparing a pharmaceutical composition that is generally non-toxic, and is not biologically undesirable and includes that which is acceptable for veterinary use and/or human pharmaceutical use.

The term “excipient” means a component of a pharmaceutical product that is not the active ingredient, such as filler, diluent, carrier, and so on. A “pharmaceutically acceptable excipient” as used in the specification and claims includes either one, or more than one, such excipient.

The term “surfactant” means a component which is also known as wetting agent, that lowers the surface tension of a liquid, allowing easier spreading. Surfactants are usually organic compounds that contain both hydrophobic and hydrophilic groups, and are thus semi-soluble in both organic and aqueous solvents. By lowering the surface tension of water, surfactants enable the solution to wet a surface or compound more quickly. Surfactants are also known as amphipathic compounds.

The phrase “amorphous rimonabant” is intended to include any amorphous form of rimonabant including but not limited to amorphous rimonabant, an amorphous solid dispersion of rimonabant, and amorphous combinations of rimonabant with pharmaceutically acceptable carriers or crystallization inhibitors.

In one aspect, the present invention provides crystalline rimonabant Form C which has good flow characteristics.

In an embodiment, rimonabant is first prepared using a process as disclosed in EP0656354. Rimonabant is then converted to an acid addition salt thereof. The preferred acid for preparing the salt is selected from oxalic acid, mandelic acid, tartaric acid, citric acid, salicylic acid, fumaric acid, sulphuric acid and phosphoric acid, most preferably hydrochloric acid.

The acid addition salt or the free base is dissolved in a water miscible solvent and added dropwise to an aqueous solution of a base, suitably an inorganic base. The inorganic base used may be selected from the group consisting of sodium hydroxide, ammonium hydroxide, potassium hydroxide, sodium carbonate, sodium bicarbonate, potassium bicarbonate, potassium carbonate and the like.

The resulting suspension may then be stirred for about 3-4 hours and the solid is filtered. The resulting solid may then be washed, suitably with water, and dried under vacuum. Typically, the drying takes place below 80° C., and preferably at a temperature ranging from 55 to 60° C. to give crystalline rimonabant Form C.

In yet another aspect, the present invention provides a process for preparing rimonabant Form C wherein the acid addition salt of rimonabant is suspended in water. The pH of the suspension may be made alkaline using aqueous base, typically an aqueous inorganic base. The inorganic base may be selected from the group consisting of sodium hydroxide, potassium hydroxide, sodium carbonate, sodium bicarbonate, potassium bicarbonate, potassium carbonate and the like.

It has been observed that rimonabant acid addition salts are hydrophobic in nature and they do not form a uniform slurry in water or water containing an inorganic base, even at higher temperatures. Hence, a catalytic amount of surfactant is added to achieve a uniform suspension. The uniform suspension assists in the formation of crystalline form C of rimonabant. The resulting suspension may be stirred for about 3 to 4 hours and filtered. The resulting solid may then be washed, suitably with water, and dried under vacuum. Typically, the drying takes place below 80° C., and preferably in a temperature ranging from 55 to 60° C. to give rimonabant Form C.

The surfactant may be selected from the group of surfactants such as macrogol esters, polysorbates 20, 40, 60, 80 and 85, and mono- and diglycerides of C12-C18 fatty acids. Other examples of surfactants are C₂-C₂₀ polyhydric alcohols, glycerin, propylene glycol, polyethylene glycol, ethylene glycol dimethyl ether, tetraethylene glycol dimethyl ether, triacetin, medium chain (C6-C10) triglycerides, and polyoxyethylene sorbitan monoesters.

Crystalline rimonabant Form C according to the present invention, or amorphous rimonabant according to present invention may be converted to a pharmaceutically acceptable salt of rimonabant by known methods. The preferable salt of rimonabant is the hydrochloride salt.

Crystalline rimonabant has the tendency to form solvates with alcohols and other solvents used for crystallization, as is acknowledged in the prior art. It is difficult to remove the residual solvent by conventional methods of drying. The processes of the present invention act as very good desolvating processes for rimonabant.

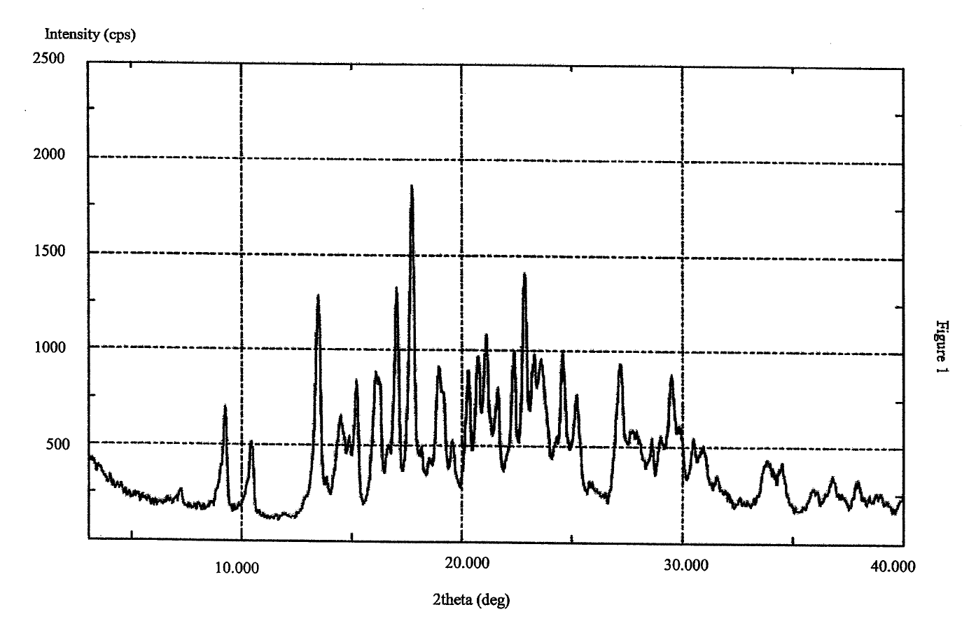

Rimonabant Form C of the present invention may be characterized by the X-ray powder diffraction spectrum as shown in FIG. 1, where the vertical axis is intensity and the horizontal axis is the 2θ angle, in degrees.

The XRPD of the rimonabant form C was measured on a Rigaku miniflex advance powder X-ray Powder Diffractometer using a Cu K alpha-1 radiation source. The peaks in the XRPD for Form C are listed in Table 1.

TABLE 1
Form C XRPD Peaks - degrees 2θ

8.82
9.25
10.42
13.45
14.49
14.84
15.18
16.09
16.23
17.0
17.7
18.9
19.12
19.52
20.25
20.73
21.11
21.60
22.37
22.82
23.27
23.56
23.74
24.55
25.18
27.15
27.7
29.47
29.91
30.46

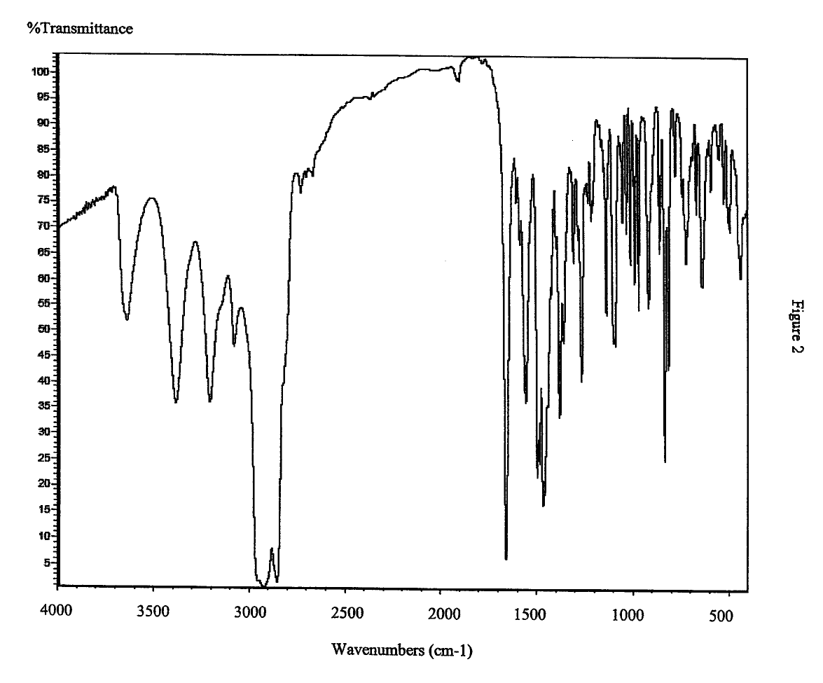

Rimonabant Form C of the present invention may be characterised by the IR spectrum shown in FIG. 2.

Rimonabant Form C of the present invention may be characterised by having characteristic IR spectra peaks at about 3639 cm⁻¹, 3388 cm⁻¹, 3207 cm⁻¹, 3079 cm⁻¹, 2806 cm⁻¹, 1556 cm⁻¹, 1265 cm⁻¹, 1138 cm⁻¹, 918 cm⁻¹, 634 cm⁻¹, 2 cm⁻¹.

Rimonabant Form C of the present invention may be characterised by having a moisture content ranging from 1% to 5%, preferably between 3.5% to 4.5%.

According to another aspect the present invention, there is provided a stable amorphous rimonabant which is substantially non-hygroscopic and has good flow characteristics.

The present invention also provides a process for the preparation of amorphous rimonabant.

Crystalline rimonabant may be prepared using the process as disclosed in EPO 656354. The crystalline rimonabant is then converted to an acid addition salt of rimonabant. The preferred acid for preparing the salt is selected from oxalic acid, mandelic acid, tartaric acid, citric acid, salicylic acid fumaric acid, sulphuric acid, phosphoric acid preferably hydrochloric acid.

The acid addition salt of rimonabant is suspended in water and stirred, suitably at a temperature below 40° C.

The pH of the suspension is made alkaline using an aqueous base such as aqueous ammonia. This step may be carried out in the absence of a surfactant. The resulting suspension is stirred, suitably for 2 to 3 hours at a temperature ranging from 25 to 30° C. and filtered. The resulting solid may be washed with water and dried under vacuum. Typically, the drying takes place below 80° C., preferably at a temperature ranging from 55-60° C. to give amorphous rimonabant.

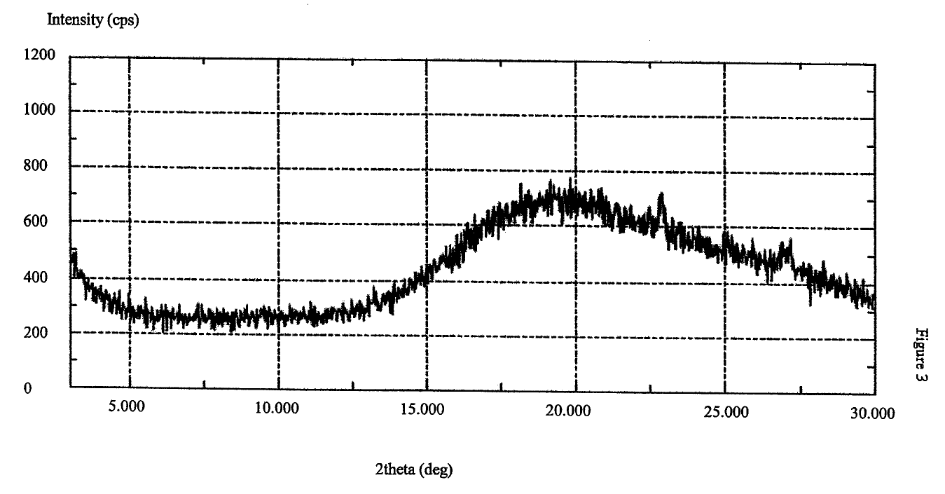

Amorphous rimonabant of the present invention may be characterized by the X-ray powder diffraction (XRPD) spectrum shown in FIG. 3, where the vertical axis denotes intensity and the horizontal axis denotes the 2θ angle, in degrees.

The XRPD of the amorphous rimonabant was measured on a Rigaku miniflex advance powder X-ray Powder Diffractometer using a Cu K alpha-1 radiation source.

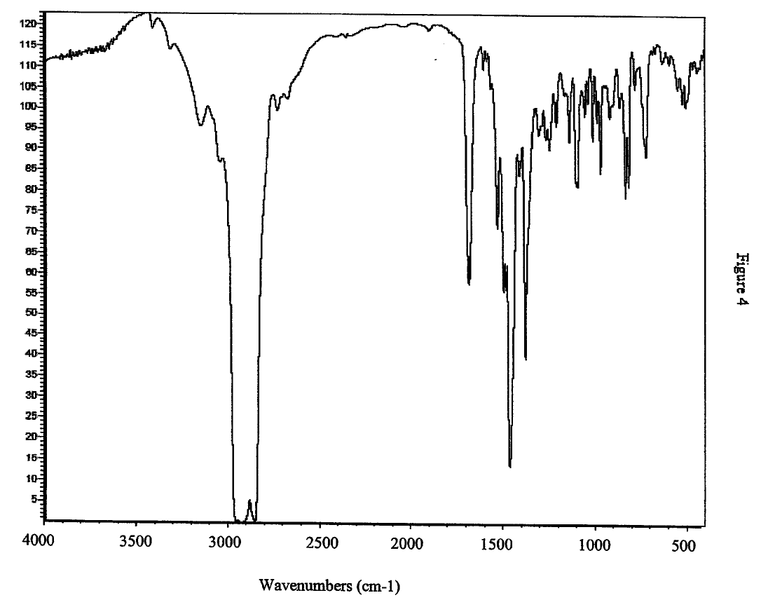

Amorphous rimonabant of the present invention may be characterized by the IR spectrum shown in FIG. 4.

Amorphous rimonabant of the present invention may becharacterized by having characteristic IR spectra peaks at about 3407 cm⁻¹, 3305 cm⁻¹, 3144 cm⁻¹, 3049 cm⁻¹, 2937 cm⁻¹, 2803 cm⁻¹, 1442 cm⁻¹, 1409 cm⁻¹, 1245 cm⁻¹, 863.60 cm⁻¹, ±2 cm⁻¹.

In another aspect, the present invention provides a pharmaceutical composition comprising rimonabant form C and one or more pharmaceutical excipients. In another aspect, the present invention provides a pharmaceutical composition comprising amorphous rimonabant and one or more pharmaceutical excipients.

The pharmaceutical compositions may be prepared by uniformly admixing the active ingredient with liquid or solid excipients and then shaping the product into the desired form. The pharmaceutical compositions may be in the form of suspensions, solutions, elixirs, aerosols, or solid dosage forms. Because of their ease of administration, tablets and capsules represent a more advantageous oral dosage unit form, in which case solid pharmaceutical excipients are employed.

Pharmaceutical excipients that can be used for the preparation of pharmaceutical compositions include, but are not limited to, hydrophilic polymers such as polyvinylpyrrolidone, gums, cellulose derivatives, cyclodextrins, gelatins, hypromellose phthalate, sugars, polyhydric alcohols, polyethylene glycol, polyethylene oxides, polyoxy ethylene derivatives, polyvinyl alcohol, propylene glycol derivatives etc. The use of mixtures of more than one of the pharmaceutical carriers to provide desired release profiles or for the enhancement of stability is within the scope of this invention.

Also, all viscosity grades, molecular weights, commercially available products, their copolymers, and their mixtures are all within the scope of this invention without limitation. Those having skill in the pharmaceutical formulation art are well aware of numerous suitable excipients, and useful procedures for making pharmaceutical compositions. The invention is further described by reference to the following examples, which set forth in detail certain aspects and embodiments of the preparation of compounds and compositions of the present invention. It will be apparent to those skilled in the art, that many modifications, both to materials and methods can be practiced without departing from the purpose and intent of this invention. The examples that follow are not intended to limit the scope of the invention as described hereinabove or as claimed below.

### EXAMPLES

### Example 1

### Preparation of Rimonabant

Crystalline rimonabant was prepared using the process disclosed in EP EP0656354.

### Example 2

### Preparation of Rimonabant Hydrochloride

Crystalline rimonabant (100 gms) was dissolved in ethyl acetate (1.0 ltr.) and stirred at 25-30° C. To the reaction mass HCl gas dissolved in isopropyl alcohol was added dropwise at room temperature until the pH of the reaction mass was 2-3. The resulting precipitate was stirred at 25-30° C. for 2 hours and filtered. The solid was washed with ethyl acetate and dried under vacuum at 55-60° C. to obtain rimonabant hydrochloride (100 gms).

### Example 3

### Preparation of Rimonabant Form C

Rimonabant hydrochloride (100 gms) was dissolved in methanol (1000 ml) and added slowly to an aqueous ammonia solution (2000 ml) at 25-30° C., and stirred. The resulting slurry was stirred for 4 hours at 25-30° C. filtered and washed with water (300 ml). The solid was dried in a vacuum oven at 60° C. for 12-14 hours to obtain rimonabant Form C (96 gms).

### Example 4

### Preparation of Rimonabant Form C

Rimonabant hydrochloride (50 gms) was dissolved in ethanol (500 ml) and added slowly to 10% sodium carbonate solution (1000 ml) at 25-30° C. and stirred. The resulting slurry was stirred for 4 hours at 25-30° C. filtered and washed with water (150 ml). The solid was dried in vacuum oven at 60° C. for about 12-14 hours to obtain rimonabant Form C (94 gms).

### Example 5

### Preparation of Rimonabant Form C

Crystalline rimonabant (100 gms) was dissolved in methanol (1000 ml) and added slowly to an aqueous ammonia solution (2000 ml) at 25-30° C., and stirred. The resulting slurry was stirred for 4 hours at 25-30° C. filtered and washed with water (300 ml). The solid was dried in a vacuum oven at 60° C. for 12-14 hours to obtain rimonabant Form C (95 gms).

### Example 6

### Preparation of Rimonabant Form C

Crystalline rimonabant (50 gms) was dissolved in acetone (500 ml) and added slowly to an aqueous ammonia solution (1000 ml) at 25-30° C., and stirred. The resulting slurry was stirred for 4 hours at 25-30° C. filtered and washed with water (200 ml). The solid was dried in a vacuum oven at 60° C. for 12-14 hours to obtain rimonabant Form C (45 gms).

### Example 7

### Preparation of Rimonabant Form C

Rimonabant hydrochloride (100 gms) was suspended in water (150 ml) containing polyoxyethylene sorbitan monooleate (0.5 gms) at 25-30° C. The pH of the reaction mass was adjusted to 9 using 10% sodium hydroxide solution. The resulting uniform slurry was stirred for 4 hours at 25-30° C. filtered and washed with water (150 ml). The solid was dried in a vacuum oven at 60° C. for 12-14 hours to obtain rimonabant Form C (95 gms).

### Example 8

### Preparation of Rimonabant Form C

Rimonabant hydrochloride (50 gms) was suspended in water (50 ml) containing polyoxyethylene sorbitan monooleate (0.05 gms) at 25-30° C. The pH of the reaction mass was adjusted to 9 using 10% potassium hydroxide solution. The resulting uniform slurry was stirred for 4 hours at 25-30° C. filtered and washed with water (150 ml). The solid was dried in vacuum oven at 60° C. for 12-14 hours to obtain rimonabant Form C (40 gms).

### Example 9

### Preparation of Rimonabant Form C

Rimonabant fumarate (50 gms) was suspended in water (50 ml) containing polyoxyethylene sorbitan monostearate (0.05 gms) at 25-30° C. The pH of the reaction mass was adjusted to 9 using 10% sodium hydroxide solution. The resulting uniform slurry was stirred for 4 hours at 30-40° C. filtered and washed with water (150 ml). The solid was dried in a vacuum oven at 60° C. for 12-14 hours to obtain rimonabant Form C (35 gms).

### Example 10

### Preparation of Amorphous Rimonabant

Rimonabant hydrochloride (100 gms) was suspended in water (1500 ml) at 25-30° C. and stirred. The pH of the reaction mass was adjusted to 9 using 10% liquor ammonia solution. The resulting slurry was stirred for 4 hours at 25-30° C., filtered and washed with water (500 ml). The solid was dried in a vacuum oven at 60° C. for 12-14 hours to obtain amorphous rimonabant (90 gms).

### Example 11

### Preparation of Amorphous Rimonabant

Rimonabant hydrogensulphate (50 gms) was suspended in water (1000 ml) at 25-30° C. and stirred. The pH of the reaction mass was adjusted to 9 using 10% liquor ammonia solution. The resulting slurry was stirred for 4 hours at 25-30° C., filtered and washed with water (500 ml). The solid was dried in a vacuum oven at 60° C. for 12-14 hours to obtain amorphous rimonabant (35 gms).

### Example 12

### Polymorph Comparison

A comparison of crystalline rimonabant Form I (prior art), crystalline rimonabant Form C (present invention) and amorphous rimonabant (present invention) was undertaken. The results were as follows.

TABLE 2
Compilation Of Onset Peaks In DSC Pattern

| FORM I | FORM C | Amorphous |
|---|---|---|
| CRD-182-109 | CRD-182-163 | CRD-251-67 |
| 155.05° C. | ~57° C. | 79.89° C. |
| | 102.74° C. | ~191.92 |

TABLE 3
Morphology

| FORM I | FORM C | Amorphous |
|---|---|---|
| CRD-182-109 | CRD-182-163 | CRD-182-181 |
| ROD SHAPED | BLOCK SHAPED | DEFINITE CLEAR |
| CRYSTALS | CRYSTALS | CRYSTALS WERE |
| | | NOT OBSERVED |

### Example 11

### Comparative Intrinsic Dissolution Study

It has been found that the order of intrinsic dissolution was amorphous >Form C >Form I.

Intrinsic dissolution was studied using an HPLC-UV method with the following parameters:

Dissolution media:—900 ml of 1% Sodium Lauryl Sulphate (SLS) in buffer.

Buffer: 0.05 M NaH₂PO₄ pH 6.6 with NaOH

Speed: 50 RPM

Temp: +37° C.

Wavelength: +240 nm

Mobile Phase:Buffer:Acetonitrile 30:70 Buffer 0.02 M NaH₂PO₄ 0.1% Triethylamine pH 2.5 with phosphoric acid.

Column: YMC Pack Pro 18 15 cm.

Flow: 1 ml/min

Pressure applied for preparation of pellet=2.5 tonns for 5 mins.

Filter=Whatman 0.45 micron.

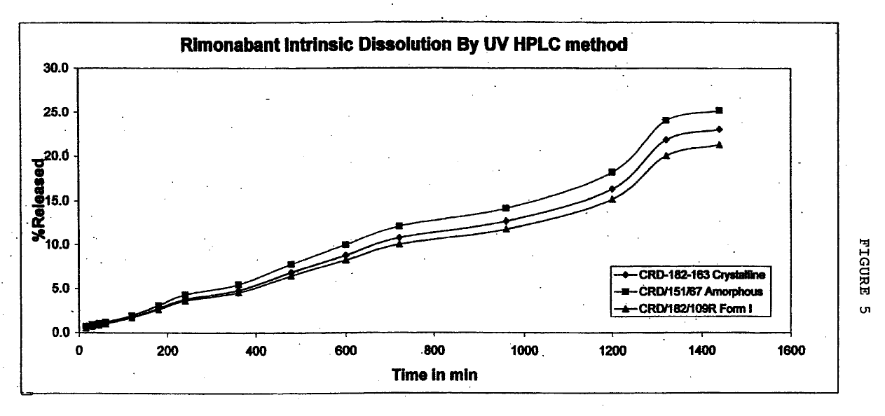

The results of the intrinsic dissolution study are illustrated in the graph in FIG. 5.

### Example 12

### Stability Testing

Long term and accelerated stability testing was carried out on crystalline rimonabant Form C.

Each sample was hermetically sealed in double HMHDPE bags and placed in fibre drum. Quantity of sample=0.8 gm per analysis. Storage conditions (long term)=30±2C., 65±5% relative humidity. Storage conditions (accelerated)=40±2° C., 75±5% relative humidity. The results were as follows.

In the long term stability testing, each sample was described as a white colour powder initially and at 3 months. The polymorphic identity was confirmed by XRPD, IR and DSC as Form C, both initially and at 3 months.

In the accelerated stability testing, each sample was described as a white colour powder initially, at 1 month, at 2 months and at 3 months. The polymorphic identity was confirmed by XRPD, IR and DSC as Form C, initially, at 1 month, at 2 months and at 3 months.

TABLE 4
Long term stability testing

| | | Chromatographic | | | |
|---|---|---|---|---|---|
| | | purity bY HPLC | | | |
| | Water | Individual | Total | | |
| | Content (By | impurity | impurities | | |
| Period of | KF) | NMT | NMT | Assay | |
| keeping | NMT 5.0% w/w | 0.10% | 1.00% | (98.0%–102.0% w/w) | Remarks |
| Initial | 3.08 | 0.08 | 0.20 | 99.86 | — |
| Months | 3.08 | 0.08 | 0.20 | 99.13 | Product is stable |

TABLE 5
Accelerated stability testing

| | | Chromatographic | | | |
|---|---|---|---|---|---|
| | Water | purity by HPLC | | | |
| | Content (By | Individual | Total | | |
| Period of | KF) | impurity | impurities | Assay | |
| keeping | NMT 5.0% w/w | NMT 0.10% | NMT 1.00% | (98.0%–102.0% w/w) | Remarks |
| Initial | 3.08 | 0.08 | 0.20 | 99.86 | — |
| 1 Month | 3.94 | 0.08 | 0.16 | 101.36 | Product is |
| | | | | | stable |
| 2 Months | 4.27 | 0.08 | 0.21 | 100.67 | Product is |
| | | | | | stable |
| 3 Months | 4.07 | 0.08 | 0.20 | 99.45 | Product is |
| | | | | | stable |

It will be appreciated that the invention may be modified within the scope of the appended claims.

What is claimed is:

1. Crystalline Form C of rimonabant.

2. The crystalline Form C of rimonabant according to claim 1, characterised by having an XRPD pattern with characteristic °2θ peaks at about 8.8, 14.5, 14.8, 21.6, 23.2, 23.6, 24.5 and 29.9±0.2 °2θ.

3. The crystalline Form C of rimonabant according to claim 2, having an XRPD pattern with further °2θ peaks at 9.3, 10.4, 13.4, 15.1, 16.1, 16.2, 17.0, 17.7, 18.9, 19.1, 19.5, 20.3, 20.7, 21.1, 22.4, 22.8, 23.7, 25.2, 27.2, 27.7, 29.5 and 30.5±0.2 °2θ.

4. The crystalline Form C of rimonabant according to claim 1, characterised by having an IR spectrum with characteristic peaks at 3639 cm⁻¹, 3388 cm⁻¹, 3207 cm⁻¹, 3079 cm⁻¹, 2806 cm⁻¹, 1556 cm⁻¹, 1265 cm⁻¹, 1138 cm⁻¹, 918 cm⁻¹, 634 cm⁻¹±2 cm⁻¹.

5. The crystalline Form C of rimonabant according to claim 1, characterised by having a moisture content ranging from 1% to 5%.

6. The crystalline Form C of rimonabant according to claim 1, having an XRPD pattern, or substantially the same XRPD pattern, as set out in FIG. 1.

7. The crystalline Form C of rimonabant according to claim 1, having an IR spectrum, or substantially the same IR spectrum, as set out in FIG. 2.

8. The crystalline Form C of rimonabant according to claim 1, having a moisture content ranging from 3.0% to 4.5%.

9. A process for preparing crystalline Form C of rimonabant comprising the steps of: (a) using an acid to convert rimonabant to an acid addition salt of rimonabant; (b) dissolving the acid addition salt of rimonabant in a water miscible solvent; (c) adding a base to the solution; (d) and isolating crystalline Form C of rimonabant.

10. A process for preparing crystalline Form C of rimonabant comprising the steps of: (a) dissolving rimonabant in a water miscible solvent; (b) adding a base to the solution; (c) and isolating crystalline Form C of rimonabant.

11. The process according to claim 9, wherein the base is an inorganic base.

12. The process according to claim 11, wherein the inorganic base is selected from the group consisting of sodium hydroxide, ammonium hydroxide, potassium hydroxide, sodium carbonate, sodium bicarbonate, potassium bicarbonate and potassium carbonate.

13. The process according to claim 9, wherein the water miscible solvent is selected from the group consisting of a C1 to C6 straight- or branched-chain alcohol, acetone and acetonitrile.

14. The process according to claim 13, wherein the water miscible solvent is methanol or ethanol.

15. A process for preparing crystalline Form C of rimonabant comprising the steps of: (a) converting rimonabant to an acid addition salt of rimonabant using the corresponding acid; (b) suspending the acid addition salt of rimonabant in a solution of water and a surfactant; (c) adding a base to the suspension; (d) and isolating crystalline Form C of rimonabant.

16. The process according to claim 15, wherein the base is selected from the group consisting of sodium hydroxide, potassium hydroxide, sodium carbonate, sodium bicarbonate, potassium bicarbonate and potassium carbonate.

17. The process according to claim 15, wherein the surfactant is selected from the group consisting of macrogol esters, polysorbates 20, 40, 60, 80 and 85, mono- and diglycerides of C12-C18 fatty acids, C₂-C₂₀ polyhydric alcohols, glycerin, propylene glycol, polyethylene glycol, ethylene glycol dimethyl ether, tetraethylene glycol dimethyl ether, triacetin, medium chain (C₆-C₁₀) triglycerides, and polyoxyethylene sorbitan monoesters.

18. The process according to claim 9, wherein the acid is selected from hydrochloric acid, oxalic acid, mandelic acid, tartaric acid, citric acid, salicylic acid, fumaric acid, sulphuric acid and phosphoric acid.

19. The process according to claim 9, wherein the crystalline Form C of rimonabant is converted to a pharmaceutically acceptable salt of rimonabant.

20. The process according to claim 19, wherein the pharmaceutically acceptable salt of rimonabant is the hydrochloride salt.

21. Crystalline Form C of rimonabant prepared according to claim 9.

22. Amorphous rimonabant.

23. The amorphous rimonabant according to claim 22, characterised by having an IR spectrum with characteristic peaks at 3407 cm⁻¹, 3305 cm⁻¹, 3144 cm⁻¹, 3049 cm⁻¹, 2937 cm⁻¹, 2803 cm⁻¹, 1442 cm⁻¹, 1409 cm⁻¹, 1245 cm⁻¹ and 863 cm⁻¹, ±2 cm⁻¹.

24. The amorphous rimonabant according to claim 22, having an XRPD pattern, or substantially the same XRPD pattern, as set out in FIG. 3.

25. The amorphous rimonabant according to claim 22, having an IR spectrum, or substantially the same IR spectrum, as set out in FIG. 4.

26. A process for preparing amorphous rimonabant comprising the steps of: (a) using an acid to convert rimonabant to an acid addition salt of rimonabant; (b) suspending the acid addition salt of rimonabant in water; (c) adding a base to the suspension; (d) and isolating amorphous rimonabant.

27. The process according to claim 26, wherein steps (b) and (c) are carried out in the absence of a surfactant.

28. The process according to claim 26, wherein the base is aqueous ammonia.

29. The process according to claim 26, wherein the amorphous form of rimonabant is converted to a pharmaceutically acceptable salt of rimonabant.

30. The process according to claim 29, wherein the pharmaceutically acceptable salt of rimonabant is the hydrochloride salt.

31. Amorphous rimonabant prepared according to claim 26.

32. A pharmaceutically acceptable salt of rimonabant prepared according to claim 19.

33. A pharmaceutically acceptable salt of rimonabant prepared according to claim 29.

34. A pharmaceutical composition comprising: crystalline Form C of rimonabant, amorphous rimonabant, or a pharmaceutically acceptable salt of crystalline Form C of rimonabant or amorphous rimonabant; and one or more pharmaceutical excipients.

35. A method of treating weight management disorders or smoking addiction in a patient in need of such treatment, which method comprises administering to the patient a therapeutically effective amount of crystalline Form C of rimonabant, amorphous rimonabant, or a pharmaceutically acceptable salt of crystalline Form C of rimonabant or amorphous rimonabant.

36. A method according to claim 35, wherein the weight management disorder is obesity.
